(19) United States
(12) Patent Application Publication
(10) Pub. No.: US 2002/0161219 A1
(43) Pub. Date: Oct. 31, 2002

(54) Non-enzymatic large scale synthesis of RNA

(76) Inventors: Anastassia Kanavarioti, Santa Cruz, CA (US); David W. Deamer, Santa Cruz, CA (US); Pierre-Alain M. Monnard, Soquel, CA (US); Claude F. Bernasconi, Santa Cruz, CA (US)

Correspondence Address: LAW OFFICES OF JONATHAN ALAN QUINE, P O BOX 458, ALAMEDA, CA, 94501

(21) Appl. No.: 09/790,440

(22) Filed: Feb. 21, 2001

(51) Int. Cl. C07H 21/02; C07H 21/04

(52) U.S. Cl. 536/25.3

(57) ABSTRACT

This invention pertains to the development of methods that accomplish efficient, non-enzymatic, nucleic acid-directed (e.g. RNA-directed) nucleic acid (e.g. RNA) synthesis. In certain embodiments the methods provide conditions that favor oligouridylate synthesis with excellent yield and, at least, up to 30% regioselectivity favoring the RNA linkage. The methods preferably involve contacting, in the presence of lead ions (Pb²⁺) and/or tin ions (Sn²⁺) and, optionally, magnesium ions (Mg²⁺), a template nucleic acid with a nucleotide derivatized with an imidazolide.

### STATEMENT AS TO RIGHTS TO INVENTIONS MADE UNDER FEDERALLY SPONSORED RESEARCH AND DEVELOPMENT

This work was supported by Grant # NCC 2-5367 from the Exobiology Program of the National Aeronautics and Space Administration administered as a cooperative grant between NASA/Ames Research Center and University of California at Santa Cruz. Support for PAM was provided by NASA grant SC-00-35. The Government of the United States of America may have certain rights in this invention.

### CROSS-REFERENCE TO RELATED APPLICATIONS

Not Applicable

### FIELD OF THE INVENTION

This invention pertains to the field of nucleic acid synthesis. In particular, this invention pertains to improved template directed chemical syntheses of nucleic acids.

### BACKGROUND OF THE INVENTION

Following publication of the antiparallel double helical structure of DNA, it became clear that base stacking stabilizes the DNA double helix and that Watson-Crick base-pairing is responsible for the complementarity of the two strands. Initially, “ex-vivo” template-directed nucleic acid synthesis was accomplished by the use various nucleic acid polymerases. In the 1980s, however, several groups began work on the possibility that polymerase enzymes and the requisite buffer systems could be replaced by simpler reaction conditions and catalysts. The concept of non-enzymatic, aqueous, template-directed (TD) oligonucleotide synthesis using chemically activated mononucleotides was pioneered by Leslie Orgel of the Salk Institute. Over the past 30 years, the Orgel group investigated non-enzymatic template-directed synthesis of polynucleotides using DNA or RNA molecules as templates. Their results indicate that bases complementary to the template sequences are preferentially incorporated into the synthesized oligonucleotide, strongly suggesting that template-directed chemical nucleic acid synthesis obeys Watson-Crick base-pairing rules.

Template-directed chemical nucleic acid syntheses, however, vary in the extent to which the full complementary product is obtained. In addition, previous template-directed methods shows problems with regioselectivity, a preference for the non-natural (2′-5′) internucleotide linkage instead of the natural (3′-5′) internucleotide linkage. In addition, the highly inefficient incorporation of uridine (or thymidine in DNA syntheses) has been a seemingly insurmountable problem that has prevented template-directed synthesis and replication with all four bases.

### SUMMARY OF THE INVENTION

This invention provides a synthetic reaction chemistry that favors oligouridylate synthesis with excellent yield and, at least, 30% regioselectivity favoring the “natural” 3′-5′ RNA linkage. The methods of this invention thus provide template-directed nucleic acid syntheses (e.g. RNA-directed RNA synthesis) in the absence of enzymes, using all four bases for nucleic acid self-replication.

In one embodiment this invention provides methods of synthesizing oligomers comprising subunits joined by phosphodiester linkages. The oligomers can be essentially any oligomer capable of adopting a double helix with a template nucleic acid. In preferred embodiments, the methods of synthesizing a nucleic acid (e.g. an RNA) involve contacting, in the presence of lead ions (Pb²⁺) and magnesium ions (Mg²⁺), or tin ions (Sn²⁺) and magnesium ions (Mg²⁺), a template nucleic acid with a nucleotide derivatized with an imidazolide.

In certain preferred embodiments, this invention provides methods of synthesizing a ribonucleic acid. The methods involve contacting a template nucleic acid, in the presence of lead ions (Pb²⁺) or tin (Sn²⁺) ions, with a nucleotide derivatized with an imidazolide. The contacting is preferably in an aqueous solution and can further comprise withdrawing water from a complex formed between the nucleotide and the template nucleic acid. In various embodiments, the withdrawing (dehydration) comprises drying said aqueous solution under a vacuum and/or freezing the aqueous solution. In particularly preferred embodiments, the nucleotide is a ribonucleoside 5′-monophosphate (e.g. a cytidylic acid, adenylic acid, guanylic acid, uridylic acid, an inosinic acid, etc.). In other preferred embodiments the nucleotide is a deoxyribonucleoside 5′-monophosphate. Preferred imidazolides include, but are not limited to imidazole (Im), 2-methylimidazole (2-MeIm), 2,4-dimethylimidazole (2,4-diMeIm), 2-aminobenzimidazole (2-aminobzIm), and 2,4,5-trimethylimidazole (2,4,5-triMeIm).

In certain embodiments, the tin and/or lead ions are present at a concentration ranging from about 0.0005 M to about 0.004M, and most preferably present at a concentration of about 0.0015 M. The derivatized nucleic acid can, optionally, also be contacted with magnesium (Mg²⁺) and/or manganese (Mn²⁺) ions. When present, the magnesium or manganese ions are present at a concentration ranging from about 0.0001 to about 0.010 M, more preferably at a concentration of about 0.005 M.

One or more of the various metal ions, when present, are preferably provided as a salt, more preferably as a salt that is not a chloride salt. In particularly preferred embodiments, one or more of the various metal ions, when present, are provided as nitrates (e.g. magnesium nitrate or manganese nitrate, lead nitrate, etc.).

In certain preferred embodiments, the ratio of template nucleic acid to mononucleotide to metal ion is about 1:1:1 where the amount of template is expressed in monomer equivalents. Where magnesium and lead (or tin) ions are both present the ratio of Mg²⁺ to lead (Pb²⁺) and/or tin (Sn²⁺) is about 4:1. When present, the template (e.g. RNA, DNA, etc.) ranges in length from about 30 to about 300 nucleotides.

In on particularly preferred embodiment the nucleotide comprises a cytodine, an adenine, a guanosine, an inosine, or a uradine; the contacting is in an aqueous solution; the tin or lead is present at a concentration of about 0.0015 M; magnesium is present at a concentration of about 0.005 M; and the template nucleic acid is present at a concentration of about 0.005M expressed in monomer equivalents. The method can further comprise freezing the mixture (e.g. for about 5 to about 30 days) or drying the mixture (e.g., for about 2 to about 6 days).

In certain embodiments, the template nucleic acid can be omitted. The methods are essentially performed as described above, but for the omission of the template nucleic acid. Thus, for example, in such embodiments, the method is a method preferably involves contacting, in the presence of lead ions (Pb²⁺) and/or tin (Sn²⁺) ions, a plurality of activated nucleotides wherein the activated nucleotides are nucleotides (e.g. ribonucleoside 5′-monophosphates, deoxyribonucleoside 5′-monophosphates, etc.) derivatized with an imidazolide (e.g., imidazole (Im), 2-methylimidazole (2-MeIm), 2,4-dimethylimidazole (2,4-diMeIm), 2-aminobenzimidazole (2-aminobzIm), 2,4,5-trimethylimidazole (2,4,5-triMeIm), etc.).

In still another embodiment, this invention provides a kit for the synthesis of a nucleic acid. In preferred embodiments, the kit comprises a container containing a nucleotide (e.g. ribonucleoside 5′-monophosphate, deoxyribonucleoside 5′-monophosphate, etc.) derivatized with an imidazolide (e.g., imidazole (In), 2-methylimidazole (2-MeIm), 2,4-dimethylimidazole (2,4-diMeIm), 2-aminobenzimidazole (2-aminobzIm), 2,4,5-trimethylimidazole (2,4,5-triMeIm), etc.). The kit optionally additionally comprises a container containing a lead salt and/or a tin salt, and/or a container containing a magnesium salt. In certain embodiments, the container containing a magnesium salt and the container containing a lead salt, and/or tin salt, are the same container. One or more of the salts, when present can be provided in an aqueous solution. In certain embodiments, one or more of the salts, when present, are not chloride salts. Particularly preferred salts, when present include nitrates (e.g. lead nitrate, magnesium nitrate, etc.). The kit can, optionally, further include instructional materials describing the synthesis of a nucleic acid according to the methods of this invention.

Definitions.

A “nucleoside” is typically a glycoside having the structure of a glycoside formed by partial hydrolysis of a nucleic acid. Nucleosides typically have either β-D ribose or β-D-2 deoxyribose in an N-glycosidic linkage between C-1 of the sugar and N-9 (purine) or n-1 (pyrimidine).

A “nucleotide” is a phosphoric ester of a nucleoside. Typically nucleotides have one or more phosphate groups esterified to the sugar. Phosphates if more than one is present, are usually attached to each other via phosphoanhydride bonds. Monophosphates can be designated as either the base monophosphate or as an -ylic acid (AMP: adenylic acid).

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included (particularly for template nucleic acids) that may have alternate backbones, comprising, for example, phosphoramide (Beaucage et al. (1993) Tetrahedron 49(10): 1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 1419), phosphorothioate (Mag et al. (1991) Nucleic Acids Res. 19:1437; and U.S. Pat. No. 5,644,048), phosphorodithioate (Briu et al. (1989) J. Am. Chem. Soc. 111:2321, O-methylphophoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp 169-176). Several nucleic acid analogs are described in Rawls, C & E News Jun. 2, 1997 page 35. These modifications of the ribose-phosphate backbone can be done to facilitate the addition of additional moieties such as labels, or to increase the stability and half-life of such molecules in physiological environments.

A “template nucleic acid” is a nucleic acid comprising a nucleotide sequence that determines/directs the nucleotide sequence of the nucleic acid synthesized according to the methods described herein. The nucleotide sequence that determines/directs the nucleotide sequence of the synthesized nucleic acid is typically complementary to the nucleic acid being synthesized.

The phrase “monomer equivalents of template” when used with reference to homopolymers, i.e. polymers that consist of one nucleotide only, refers to the equivalent amount of mononucleotide before polymerization

### BRIEF DESCRIPTION OF THE DRAWINGS

FIG. 1 illustrates various activated monomers used in the reactions described in the examples.

FIG. 2 illustrates the HPLC elution profiles of products from the metal ion-catalyzed oligomerization of ImpU. Samples with 5.2 mM of ImpU (1) were incubated at −18° C. (frozen samples). Top: Reaction carried out for 9 days in the presence of 6.1 mM Mg(NO₃)₂ and 1.5 mM Pb(NO₃)₂ at pH 6.91. Middle: Reaction carried out for 9 days in the presence of 6.1 mM Mg(NO₃)₂ at pH 7.08. Bottom: Same reaction mixture as in top profile but incubated for 31 days. Given concentrations correspond to initial solution. No other salt of buffer was added. pH's were measured at room temperature and remained stable during incubation. Presumably the imidazolide moiety of the substrate acts as the buffer. Aliquots were quenched with EDTA and kept at +10° C. in the HPLC's autosampler before HPLC analysis. The products were eluted from the RPC-5 column with a linear aqueous NaClO₄ gradient at pH 8 made with Trizma buffer (0 to 0.04 M NaClO₄ in 40 min). Product yield is expressed as the sum of the HPLC units reported under the oligomeric product peaks over the sum of the HPLC units reported for all the peaks (monomer and oligomers) that corresponds to the initial amount of substrate monomer present. Only samples with the same (±5%) total HPLC areas are compared. Oligomer yields were not corrected for hypochromicity and are underestimated by about 10%. The length of the oligouridylate products was estimated by comparison of the retention times of products from partial degradation of poly(U) chromatographed under the same conditions.

FIG. 3 shows HPLC elution profiles of products from the Mg/Pb-catalyzed oligomerization of uridine derivatives. Samples with 5.2 MM of a phosphoimidazolide activated uridine derivative incubated for 8 days at −18° C. in the presence of 6.1 mM Mg(NO₃)₂ and 1.5 mM Pb(NO₃)₂ (frozen samples). Top: 2-MeImpU (2) at pH 7.73. Middle: 2,4-diMeImpU at pH 7.69. Bottom: 2-AbzImpU at pH 7.26. Given concentrations correspond to initial solution. No other salt of buffer was added. pH's were measured at room temperature and remained stable during incubation. Presumably the imidazolide moiety of the substrate acts as the buffer. HPLC analysis, product yield determination and length estimation of oligomers were done as described in the caption of FIG. 2.

FIG. 4 shows HPLC elution profiles of products from the Mg/Pb-catalyzed oligomerization ImpA, ImpG or ImpC. Samples made with 5 mM of ImpA, ImpG or ImpC were incubated for 8 days at −18° C. (frozen samples) in the presence of 5.2 mM Mg(NO₃)₂ and 1.3 mM Pb(NO₃)₂. Top: ImpA at pH 6.43. Middle: ImpG at pH 6.40. Bottom: Impc at pH 6.70. Given concentrations correspond to initial solution. No other salt of buffer was added. pH's were measured at room temperature and remained stable during incubation. Presumably the imidazolide moiety of the substrate acts as the buffer. Aliquots were quenched with EDTA and kept at +10 ° C. in the HPLC's autosampler before HPLC analysis. The products were eluted from the RPC-5 column with an aqueous NaClO₄ gradient at pH 12 made with 0.01 M NaOH (isocratic with 0.01 M NaOH for 4 min, from 0 to 0.03 M NaClO₄ in 27 min). The length of the products was estimated by comparison of the retention times of products from partial degradation of poly(C) or poly(A) chromatographed under the same conditions. Product yield was determined as described in caption of FIG. 2.

FIG. 5 shows HPLC elution profiles of products from the metal ion-catalyzed oligouridylate synthesis in frozen samples at −18° C. Top: Sample was made with 5.2 mM of ImpU, 5.2 mM MgCl₂ and 0.65 mM of Pb(NO₃)₂ at pH 6.70 and was incubated for 34 days. Middle: Sample was made with 5.2 mM of ImpU, 5.2 mM MnCl₂ and 0.65 mM Pb(NO₃)₂ at pH 6.86 and was incubated for 30 days. Bottom: Sample was made with 5.2 mM of 2-MeImpU, 5.2 mM MgCl₂ and 4 mM SnCl₂ at pH 7.20 and was incubated for 12.8 days. No buffer was added. Sample preparation and analysis of products as described in the caption of FIG. 4. Estimation of oligomer length as described in the caption of FIG. 2.

FIG. 6 shows HPLC elution profiles of products from the Mg/Pb-catalyzed ImpU oligomerization. All samples but the one represented in the bottom profile were made with 5.2 mM ImpU. Samples were incubated for 4.8 days at indicated temperatures in the presence of 5.2 mM Mg(NO₃)₂ and 1.3 mM Pb(NO₃)₂ at pH 6.55. Reaction mixtures included 11 mM MES buffer and 8% acetonitrile to prevent microbial growth at the higher temperatures. The first and last were frozen, the others remained liquid. The first four profiles correspond to samples with 5.2 mM ImpU. The bottom profile corresponds to a sample made under identical conditions as the other four but with 0.65 mM ImpU. For the sake of visual comparison, an 8-fold larger amount of the 0.65 mM ImpU sample was used for analysis. Sample preparation and analysis of products as described in the caption of FIG. 4. Estimation of oligomer length as described in the caption of FIG. 2.

### DETAILED DESCRIPTION

This invention pertains to the development of methods that accomplish efficient, non-enzymatic, nucleic acid-directed (e.g. RNA-directed) nucleic acid (e.g. RNA) synthesis. In certain embodiments the methods provide conditions that favor oligouridylate synthesis with excellent yield and, at least, 30% regioselectivity favoring the RNA linkage.

The syntheses of this invention can be accomplished in a simple “one-pot” format. In preferred embodiments, the method entails contacting a template nucleic acid in the presence of lead (Pb²⁺) and/or tin (Sn²⁺) and one or more activated nucleotide monomers (e.g. nucleotide(s) derivatized with an imidazolide). The reaction is preferably performed in an aqueous solution and subsequent withdrawal of water results in the template directed polymerization of the activated nucleotide monomers.

Without being bound to a particular theory, it is believed that internucleotide bond formation occurs by nucleophilic substitution with one of the sugar (e.g. ribose) hydroxyls acting as the nucleophile attacking the P—N bond of another molecule. The metal ion(s) presumably stabilize the double helical structure formed between polymer and monomers and activate the ribose hydroxyls. The nucleophilic attack leads to the scission of the P—N bond, formation of the O—P bond and expulsion of the imidazole. This mechanism appears to be preferred in the presence of the template. It is believed that the template assumes a major role in directing the synthesis by coding for the complementary base and, perhaps, organizing a more ordered transition state.

More particularly, without being bound to a particular theory, we believe that the drying and freezing conditions described here limit the presence of water molecules to the ones that are structurally bonded to the metal ion and to the phosphate backbone, so that hydrogen-bonding between complementary bases is enforced. Base stacking between the bases of the polymer may also stabilize a single stranded RNA structure, e.g., as compared to random coil, thereby facilitating double helical arrangement. Moreover, under limiting water conditions both purines and pyrimidine are likely to form stacks of similar stability, leading to the surprising polymerization of uridine derivatives under the reaction conditions described herein.

It is also believed that the P—N bond in the phosphoimidazolide derivatives is much more labile than the P—O bond of the triphosphate nucleotides, i.e. the natural substrates in biological nucleic acid synthesis, and this is why the former are better substrates than the latter in the absence of enzymes. Nevertheless, the reactivity of the P—N bond leads, in the presence of water, to P—N bond hydrolysis and consequently to irreversible deactivation of the monomers. A consequence of this reactivity is that internucleotide bond formation is catalyzed preferentially over P—N bond hydrolysis.

We have designed two preferred methods that accomplish efficient non-enzymatic, RNA-directed RNA synthesis. Both involve driving the reaction by high concentrations of reactants that are achieved either by freezing (e.g. at −18° C.), or by removing water (e.g., under vacuum). Removal of water under vacuum obviously concentrates reactants, but the mechanism of concentration by freezing is less apparent. During freezing, numerous microscopic ice crystals that are virtually pure water form within the mixture. As a result, solutes are forced in the spaces between the crystals and produce highly concentrated semi-fluid eutectics within which chemical reactions can occur.

The same reaction mixture can be used in either dehydration or freezing methods. In preferred embodiments, the reaction mixture comprises the template (e.g. an RNA template), and all the necessary monomers present as phosphoimidazolide-activated derivatives. The presence of Pb²⁺ and/or Sn²⁺ greatly facilitates the polymerization. In certain preferred embodiments, the concentrations of the starting materials are initially 0.005 M each with the concentration of the template expressed in monomer equivalents. The ratio of [template] to [mononucleotide] is typically 1:1. When magnesium (Mg²⁺) is present the ration of [template] to [mononucleotide] to [magnesium ion] is typically 1:1:1. Lead (II) ion and/or tin (II) ion is added at 0.0015 M, i.e. [Mg2+]:[Pb2+]%4:1.

In preferred embodiments, the reaction mixture excludes chloride, which tends to precipitate Pb²⁺. Thus, the tin and/or lead and/or magnesium are typically added in forms other than chloride salts (e.g. they can be added as nitrates and not as the chloride salts). Despite the low initial concentration of monomers, the concentration step produces practically quantitative yields of oligomerization products. Buffers and other salts are not necessary, although sodium nitrate may increase product yields.

In one preferred embodiment, the samples are placed in a dessicator under vacuum and dried at 25 to 40 torr at room temperature. In another embodiment, the samples are frozen and kept in a bath at −18° C. Preliminary comparison between the two methods indicates that under dry conditions the reaction is complete within 5 days and under freezing conditions within 10 to 15 days. Although more sluggish, freezing leads to better efficiency and higher regioselectivity than drying.

While the synthesis methods of this invention are described with respect to the use of activated monomers, it will be appreciated that activated polymers could be used instead of or in addition to the use of activated monomers. Thus for example, a similar reaction could be used using 16 different activated nucleotide dimmers or 64 different activated nucleotide trimers. The use of activated multimers instead of or in addition to activated monomers is expected to increase fidelity of the template replication.

I. Imididazolide-derivatized nucleotides (activated monomers).

The methods of this invention can be performed with a variety of activated nucleotides although imidazolide-derivatized nucleotides are most preferred. The nucleotides include, naturally-occurring deoxyribonucleotides and ribonucleotides as well as modified and/or synthetic nucleotides. Preferred naturally-occurring nucleotides include, but are not limited to the ribonucleotide and deoxyribonucleotides forms of adenosine, guanosine, cytosine, uridine, and thymidine. Other preferred nucleotides include inosine, xanthosine, uridine, orotidine, and the like. A wide variety of derivatized and/or synthetic nucleotides are suitable for activation/derivatization with an imidazolide and used in the methods of this invention. These, include, but are not limited to 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N₆-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N₆-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N₆-isopentenyladenine, uracil-5-oxyacetic acid methylester, uracil-5-oxyacetic acid, oxybutoxosine, pseudouracil, queosine, 2-thiocytosine, 5 methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, 2,6-diaminopurine, and the like.

A great number of imidzaolides can be used to activate/derivatize the nucleotide monomers for use in the methods of this invention. Such imidazolides include, but are not limited to imidazole (Im), 2 methyl imidazole (2-MeIM), 2,4-dimethylimidazole (2,4,diMeIM), 2-aminobenzimidazole, 2-ethyl-4-methylimidazole, 1-(2-cyanoethyl)-2-ethyl-4-methylimidazole, 2-n-butyl-imidazole, 2-nitroimidazole, 4-nitroimidazole, and the like. In a particularly preferred embodiment, the inidazolides include imidazole (Im), 2 methyl imidazole (2-MeIM), 2,4-dimethylimidazole (2,4,diMeIM), 2-aminobenzimidazole, purine derivatives, such as adenine derivatives and the like. Table 1 illustrates certain preferred imidazole-derivatized nucleotides used in the methods of this invention.

TABLE 1
Derivatized nucleotides for template-directed nucleic acid synthesis
(abbreviations)

| Abbreviation | Derivatized Nucleotide |
|---|---|
| ImpA | adenosine-5′-monophosphate-imidazolide |
| ImpU | uridine-5′-monophosphate-imidazolide |
| ImpG | guanosine-5′-monophosphate-imidazolide |
| ImpC | cytidine-5′-monophosphate-imidazolide |
| ImpI | inosine-5′-monophosphate-imidazolide |
| 2-MeImpA | adenosine-5′-monophosphate-2-methylimidazolide |
| 2-MeImpU | uridine-5′-monophosphate-2-methylimidazolide |
| 2-MeImpG | guanosine-5′-monophosphate-2-methylimidazolide |
| 2-MeImpC | cytidine-5′-monophosphate-2-methylimidazolide |
| 2-MeImpI | inosine-5′-monophosphate-2-methylimidazolide |
| 2,4-diMeImpA | adenosine-5′-monophosphate-2,4-dimethylimidazolide |
| 2,4-diMeImpU | uridine-5′-monophosphate-2,4-dimethylimidazolide |
| 2,4-diMeImpG | guanosine-5′-monophosphate-2,4-dimethylimidazolide |
| 2,4-diMeImpC | cytidine-5′-monophosphate-2,4-dimethylimidazolide |
| 2,4-diMeImpI | inosine-5′-monophosphate-2,4-dimethylimidazolide |
| 2-NH₂bzImpA | adenosine-5′-monophosphate-2-aminobenzimidazolide |
| or 2-AbzImpA | |
| 2-NH₂bzImpU | uridine-5′-monophosphate-2-aminobenzimidazolide |
| or 2-AbzImpU | |
| 2-NH₂bzImpG | guanosine-5′-monophosphate-2-aminobenzimidazolide |
| or 2-AbzImpG | |
| 2-NH₂bzImpC | cytidine-5′-monophosphate-2-aminobenzimidazolide |
| or 2-AbzImpC | |
| 2-NH₂bzImpI | inosine-5′-monophosphate-2-aminobenzimidazolide |
| or 2-AbzImpI | |
| 2,4,5-triMeIm | 2,4,5-trimethylimidazole |

The nucleotides can be derivatized with the imidazole(s) according to any of a number of methods well known to those of skill in the art. In one preferred embodiments, the activated monomers are produced by oxidative condensation of the imidazolide and the nucleoside 5′monophosphate in anhydrous solvents. Preferred protocols can be found in Joyce et al. (1984) J. Mol. Biol. 176: 279 Kanavarioti et al. (1995) J. Org. Chem. 60: 632-637, and in the examples provided herein. The monomer preparations synthesized, e.g. as described herein were 96 to 98% pure and obtained in practically quantitative yield. These nucleotide derivatives are solids, very stable under dry/cold conditions and relatively stable under dry/room temp. conditions.

II. The Catalyst (Pb²⁺)

In preferred embodiments, the methods of this invention utilize lead (Pb²+) as a “catalyst”. In particular, the combination of Mg²⁺ and Pb²⁺ was found to facilitate polymerization selectively over hydrolysis. Pb²⁺ also works alone as well as in conjunction with a number of other metals such as Mn²⁺. Tin ions (Sn²⁺) also work almost as efficiently as lead ions alone or in the presence of Mg²⁺. It is noted, however, that a small number of other metals and metal combinations were tested unsuccessfully. Among them were Mn²⁺, Zn²⁺, Mg²⁺, Mg²⁺/Mn²⁺ and Mg²⁺/Zn²⁺.

In preferred embodiments, species that drive lead out of aqueous solution are preferably avoided. Thus, for example, preferred reaction mixtures exclude chloride (Cl⁻). Thus, the lead, magnesium, or other metals, are provided in a form other than a chloride salt. Such forms include nitrates, phosphates, and the like. In one most preferred embodiment, the lead, and/or tin, and/or magnesium are provided as nitrates.

III. The template.

The experiments described herein show that the double helical structure is still in place and still sensitive to its environment in the absence of bulk water both at room temperature and at −18° C. Thus, we believe that any informational polymer that forms stable helical structures with its complement could, in principle, be copied using the methods described herein. Thus, the template directed synthesis methods described herein can be performed with a number of different templates including, but not limited to deoxyribonucleic acids (DNA), ribonucleic acids (RNA), peptide nucleic acids (PNA), and the like. The template can comprise “naturally-occurring” monomers or modified monomers as long as the modified monomers still permit the ordering and formation of a double helix with the synthesized nucleic acid.

The templates can be virtually any length. In certain embodiments, the templates range in length from about 20 to about 1000 monomers (e.g. nucleotides), preferably from about 30 to about 500 monomers, more preferably from about 50 to about 300 monomers. It is believed that the longer the template the more stable the double helical polymer/monomer complex and the more efficient the polymerization. Thus, we believe that the present methodology will be best suited for the synthesis of oligomers in the range of 30 to 100 monomers (e.g. nucleotides) long.

The template can be provided in solution or it can be attached to a solid substrate. The use of a solid-phase bound template allows ready separation of the template from the reaction mixture. In addition, the same template could be used repeatedly in new synthesis cycles in order to obtain several fold yield of product from a relatively small amount of template

A preferred solid support preferably withstands the reaction chemistry, does not interfere with the synthesis, and permits direct or indirect (e.g. via a linker) attachment of the template nucleic acid. Suitable substrates include, but are not limited to glass, plastic, silicon, minerals (e.g. quartz), semiconducting materials, ceramics, metals, metalloids, semiconductive materials, cermets or the like. In addition, substances that form gels can be used. Such materials include, e.g., proteins (e.g., gelatins), lipopolysaccharides, silicates, agarose and polyacrylamides. Where the solid surface is porous, various pore sizes can be employed depending upon the nature of the system.

In preparing the surface, a plurality of different materials cab be employed, particularly as laminates, to obtain various properties. For example, proteins (e.g., bovine serum albumin) or mixtures of macromolecules (e.g., Denhardt's solution) can be employed to avoid non-specific binding, simplify covalent conjugation, and the like. If covalent bonding between a template and the surface is desired, the surface will usually be polyfunctional or be capable of being polyfunctionalized. Functional groups which cab be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature.

For example, methods for immobilizing nucleic acids by introduction of various functional groups to the molecules is known (see, e.g., Bischoff (1987) Anal. Biochem., 164: 336-344; Kremsky (1987) Nucl. Acids Res. 15: 2891-2910). Modified nucleotides can be placed on the template using PCR primers containing the modified nucleotide, or by enzymatic end labeling with modified nucleotides. Use of glass or membrane supports (e.g., nitrocellulose, nylon, polypropylene) is advantageous because of well developed technology in this area.

Alternatively, templates can also be immobilized on commercially available coated beads or other surfaces. For instance, biotin end-labeled nucleic acids can be bound to commercially available avidin-coated beads. Streptavidin or anti-digoxigenin antibody can also be attached to silanized glass slides by protein-mediated coupling using e.g., protein A following standard protocols (see, e.g., Smith (1992) Science 258: 1122-1126). Biotin or digoxigenin end-labeled nucleic acids can be prepared according to standard techniques. Alternatively, paramagnetic particles, such as ferric oxide particles, with or without avidin coating, can be used.

A typical HPLC elution profile indicated that the monomer elutes early, the polymer elutes last and the oligomer products elute in between the monomer and the polymer peaks. The formation of a series of oligomer products with increasing length is a typical result of reactions run with long homopolymers as templates (˜300 bases long). This is because initiation of the polymerization can occur at any site on the template and consequently the products vary in length. In order to produce the full complement one may accompany the synthesis step by an additional activation/ligation step. Thus, for example, in certain embodiments with the relatively long templates, product oligomers might be truncated. In order to produce the full complement the synthesis cycle could be repeated with fresh activated nucleotide(s) or a condensing agent such as water-soluble 1,1′-carbonyl-diimidazole derivative could be added to the mixture.

Another system better suited for the synthesis of the template complement is a preformed hairpin with two arms, one short and one long that includes the template of interest. Synthesis in this system starts as elongation of the short arm that is faster than initiation at any other site in between. Template-directed synthesis using hairpins has been applied successfully in solution to produce the full complement of sequences devoid of uridine. Thus hairpin oligonucleotides can produce a good yield of the full complement. The hairpin can then be removed using methods well known to those of skill in the art (e.g. cleavage with restriction enzymes). Alternatively, the hairpin could be removed by strategically placing a functionalized nucleotide or a pyrophosphate bond at a position that does not interfere with the hairpin structure and selectively inducing cleavage of the product after the completion of the synthesis cycle.

IV. Reaction conditions/optimization.

The methods of this invention are easily accomplished in an aqueous system. No buffers are required and the activated monomers (e.g. nucleotides) need not be protected. The reactions can be run in a buffered system as long as the buffer components do not precipitate Pb²⁺, Sn²⁺, or Mg²⁺, when present, and further do not interfere with the template-directed synthesis.

In preferred embodiments, the reaction is run at a pH ranging from about pH 5 to about pH 7.5, preferably from about pH 5.5 to about pH 7, and most preferably from about pH 6.0 to about pH 7.

In preferred embodiments, the lead or tin concentration ranges from about 0.0003 M to about 0.006 M, more preferably from about 0.0004 M to about 0.005 M, and most preferably from about 0.0005 M to about 0.004M, and, in one particularly preferred embodiment is about 0.0015 M.

When present, the magnesium concentration ranges from about 0.001 M to about 0.01M, and most preferably from about 0.003 M to about 0.006 M. In one preferred embodiment, the magnesium is present at a concentration of about 0.005 M.

In preferred embodiments, the ratio of template nucleic acid to mononucleotide to metal ion is about 1:1:1 where the amount of template is expressed in monomer equivalents.

In certain embodiments, the ratio of Mg²⁺ to Pb²⁺ (or Sn²⁺) ([Mg2+]:[Pb2+]%)ranges from about 10:1 to about 1:1, more preferably from about 6:1 to about 1:1, and most preferably from about 5:1 to about 1:1. In one preferred embodiment the Mg2⁺ to Pb²⁺ ratio is about 4:1.

The reaction mixture can be dehydrated according to any of a number of methods well known to those of skill in the art. In one particularly preferred embodiment, the reaction mixture is vacuum desiccated. Thus, for example the reaction mixture samples are placed in a dessicator under vacuum and dried at 25 to 40 torr at room temperature. In another embodiment, the reaction mixture samples are frozen (e.g. frozen and kept in a bath at −18° C.).

Other methods of dehydration are also acceptable. Thus, for example, freezing can be combined with vacuum drying (e.g. lyophilization). In other embodiments, dehydration can be effected by the dialyzation, or displacement of water with a series of organic solvents.

It is believed that routine optimization of the described methodology can lead to faster reactions, higher efficiency and even better regioselectivity. Parameters available for optimization include the pH of the original solution, the nature of the leaving group, the nature of the metal ion(s) and the relative and absolute concentrations of the reagents in the reaction mixture. In addition, under dry conditions, humidity may play a role.

With Mg²⁺/Pb²⁺ degradation of the templates is detectable at long incubation times. This degradation can be suppressed by using conditions/parameters that expedite the polymerization reaction. Experiments suggest that the rate of polymerization is controlled by the pH of the aqueous solution before freezing or drying and by the imidazole derivative. Specifically, the reactivity follows the order Im→2-MeIM→2,4-diMeIm→2-AbzIm-. The optimal pH seems to be in the range 6.0<pH<7.0. In addition, the concentration of the metal ions should preferably be kept at a minimum level, e.g. 0.003 M <[Mg²⁺] ¾ 0.006 M and 0.001 M<[Pb²⁺]<0.002 M.

Each one of the reactions described herein can be optimized within a sequence of nucleotides, preferably adenines, present in the template strand. Cooptimization might be feasible, unless a metal ion is found to specifically inhibit a certain reaction. Other areas that can be optimized reaction product purification and removal of metal ions by mixing with a solid-phase bound EDTA complex and filtering.

It is clear from the data presented herein that nucleic acid (e.g. RNA)-directed nucleic acid (e.g. RNA) synthesis can be readily accomplished using the methods described herein. In addition, the experiments described herein demonstrate that the double helical structure is still in place and still sensitive to its environment in the absence of bulk water both at room temperature and at -18° C. This suggests that any informational polymer that forms stable helical structures with its complement can, in principle, be copied using one of our methods. This opens the way to template-directed synthesis of polynucleotide analogues using RNA or DNA templates, as long as the former makes a double helical structure with one of the latter.

V. Product cleanup.

Reaction products can be cleaned up (purified) according to standard methods for nucleic acid purification well known to those of skill in the art. Such methods include, but are not limited to gel electrophoresis, HPLC, capillary electrophoresis, FPLC, affinity chromatography, and the like.

In one preferred embodiment, oligomers are cleaned up by HPLC using an RPC5 column packed with RPC5. RPC5 is a certain type of KEL F that is coated with Adogen, a commercially available quaternary ammonium salt. RPC5 resolves oligomers according to length, base composition and linkage and so far has shown unparalleled resolving power for short to medium range oligonucleotides. A similar column, DNAPac PA-100 supplied by Dionex, although not tested by us, claims to have resolving capabilities comparable to RPC5. Monomers, oligomers as well as polymers elute from RPC5 using a sodium perchlorate (0.1 M NaClO₄) gradient at pH 8 (tris.HClO₄ buffer) for oligo(A), oligo(C) and oligo(U) analysis or at pH 12 (0.01 M NaOH) for oligo(G) analysis.

Because products resulting from degradation of the template also elute in the same region as the polymerization products, it is desirable to use controls and product identification/verification to ensure that the peaks are the desired polymerization products rather than degradation products. The following analytical methods were used or are recommended for the identification of the peaks eluting in the middle of the HPLC profile (polymerization or degradation products). These are (1) UV-vis spectroscopy using the diode-array detector of the HPLC instrument used for analysis. This was done with C/G samples, but not with U/A samples because of the similarity of the spectra of the latter pair. (2) NaOH degradation leads to cleavage of both internucleotide bonds yielding the corresponding monomer. The monomer was identified by analysis with C18 chromatography/diode array detection and coelution with standards using HPLC methods developed in our laboratory. NaOH degradation was done with isolated fractions of oligomer products. (3) Selective enzymatic degradation of the products with RNase A successfully distinguishes oligouridylates from poly(A) decomposition products and with Ribonuclease T1 oligoguanylates from poly(C) degradation products. Because the 2′-5′-internucleotide linkages can not be cleaved enzymatically, enzymatic degradation of the products with Nuclease P1 provided information about the extent of regioselectivity. (4) Mass spectroscopy can, but has not yet, been used to establish oligomer length from selected oligomer fractions.

VI. Kits.

In another embodiment, this invention provides kits for the synthesis of a nucleic acid. The kits preferably comprise a container containing a nucleotide derivatized with an imidazolide. Optionally included is a container containing a magnesium salt; and/or a container containing a lead salt. The kits can also, optionally include, instructional materials teaching the use of a derivatized nucleotide (e.g. an imidazole-derivatized nucleotide) in conjunction with lead, and optionally magnesium for use in the template directed synthesis of a nucleic acid (e.g. an RNA).

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips (e.g. FLASH ROM)), optical media (e.g., CD ROM), and the like. Such media may include addresses to internet sites that provide such instructional materials.

### EXAMPLES

The following examples are offered to illustrate, but not to limit the claimed invention.

### Example 1

### Synthesis of the Nucleoside Phosphoimidazolide Derivatives

Protocol for High performance Liquid Chromatography (HPLC)

Samples were thawed to room temperature and aliquots were analyzed by HPLC using an RPC5 column packed in our laboratory with RPC5. RPC5 is a certain type of KEL F and that was coated with Adogen, a commercially available quarternary ammonium salt. RPC5 resolves oligomers according to length, base composition and linkage and so far has shown unparalleled resolving power for short to medium range oligonucleotides. A similar column, DNAPac PA-100 supplied by Dionex, although not tested by us, claims to have resolving capabilities comparable to RPC5. Monomers, oligomers as well as polymers elute from RPC5 using a sodium perchlorate (0.1 M NaClO₄) gradient at pH 8 (tris.HClO₄ buffer) for oligo(A), oligo(C) and oligo(U) analysis or at pH 12 (0.01 M NaOH) for oligo(G) analysis.

Protocol for the Synthesis of the Nucleoside Phosphoimidazolide Derivatives (see Table 1)

The following can be found in “Relative Reactivity of Ribosyl 2′-OH vs. 3′-OH in Concentrated Aqueous Solutions of Phosphoimidazolide Activated Nucleotides” by A. Kanavarioti, L. F. Lee and S. Gangopadhyay, Origins of Life and Evolution of the Biosphere 29, 473-487, 1999.

Materials.

Reagent grade chemicals were used throughout. Solvents were HPLC quality. Nucleoside 5′-monophosphates of adenosine, cytidine, guanosine, inosine and uridine (5′ NMP with N=A, C, G, I or U) in the free acid form were purchased from Sigma. Imidazole and 2-methylimidazole and 2-aminobenzimidazole (Aldrich) were recrystallized from toluene using hot filtration. 2, 4-dimethylimidazole (about 90% pure from ACROS/Fisher) was dissolved in the least possible volume of toluene, the solution was dried with MgSO₄, filtered and added dropwise to petroleum ether at room temperature from which 2,4-dimethylimidazole precipitated after long and vigorous stirring.

The sodium or lithium salts of the listed compounds were synthesized based on the method developed by Mukaiyama and Hashimoto (1971) Bull. Chem. Soc. Japan, 44: 2284. The actual procedure has been reported elsewhere (see, e.g., Joyce et al. (1984) J. Mol. Biol. 176: 279; Kanavarioti et al. (1995) J. Org. Chem. 60: 632-637, 1995, see the protocol for synthesizing 2-MeImpG which is also detailed below). The materials, 97% pure or higher, contain about 1% each of 5′NMP and of the pyrophosphate dinucleotide based on high performance liquid chromatography (HPLC). All substrates were white precipitates. 2,4-diMeIm-derivatives were also synthesized following the above method, only that they were about 85% pure. One of the impurities is about 11% of the 2-MeIm-derivative, most likely formed by 2-methylimidazole contamination in 2,4-dimethylimidazole. In addition, condensation between 2,4-dimethylimidazole and the nucleotide yielded, as expected, two products: the 2,4-diMeImpN and the 2,5-diMeImpN.

The preparation contained the two isomers in a ratio of about 10:1, presumably 2,4-diMeImpN being the major product. Steric congestion between the 5-position of the imidazole and the phosphate oxygen most likely disfavors the synthesis of the 2,5-diMeImpN compared to the synthesis of the 2,4-diMeImpN. Molecular weight determination for both isomers was performed by means of a mass spectrometer by Micromass Inc. equipped with an 1100 HP liquid chromatograph (LC/MS). These preparations were used without further purification.

The success of the synthesis in both yield (about 90%) and purity can be attributed to the strict adherence to the following procedures. All materials used for the synthesis were either freshly distilled or dried over phosphorus pentoxide at 4 mmHg pressure overnight. Reaction and work-up was done in an argon atmosphere. Reaction time was kept at 1 h, because longer times resulted in increased yields of the pyrophosphate by-product. Filtration was accomplished under a stream of dry argon and controlled reduced pressure. The precipitate was washed well with ether/acetone before letting it dry in the argon stream. Especially the uridine derivatives tend to absorb moisture and get easily liquified before they are well washed. Once the product is washed and dried under argon, it can be handled without special care. Typical problems that compromise purity, as indicated by low extinction coefficients are: Coprecipitation of NaClO₄ and formation of a mixture of sodium and triethylammonium salts of the nucleotide derivative. Purification is accomplished by dissolving the dry substrate in DMSO (10 mL), filtering it with paper and precipitate it dropwise in a ether/acetone solution (100 mL) that includes 0.5 mL of NaClO₄ saturated acetone solution. The sodium salts of ImpA, 2-MeImpA can be purified by dissolving them in DMF. However, cytidine derivatives are insoluble both in DMSO and DMF.

Synthesis of the sodium salt of 2-MeImpG: 5′-GMP free acid, 0.46 g (1.25 mmol), and 1.64 g of 2-methylimidazole were dissolved with stirring in approximately 10 mL of dimethylsulfoxide in a 50 mL round bottom flask. Four hundred mL of freshly distilled triethylamine was added, followed by 1.05 g (4 mmol) of triphenylphosphine and 0.88 g (4 mmol) of f2,2′-dipyridyldisulfide. The mixture was stirred for I h and quenched by adding dropwise with stirring to a 250 mL Erlenmeyer flask cooled at 0° C. containing 50 mL of acetone, 50 mL of anhydrous ether, and 4 mL of saturated sodium perchlorate/acetone. Sodium is replaced by lithium perchlorate for the synthesis of the corresponding lithium salt. This was stirred for 20 min to allow precipitation and the product collected by vacuum filtration and washed three times with 1:1 acetone: ether and three times with acetone. The product was dried under reduced pressure and purified if necessary as described below.

### Example 2

### Template-Directed Synthesis of RNA

Experiments were performed by mixing ribopolymers with ribomononucleotides and then adding the metal ion(s). Five ribonucleotides (uridine (U), cytidine (C), guanosine (G), adenosine (A) and inosine (I)) in the form of a phosphoimidazolide activated derivative have been used as monomers. Imidazole (Im), 2-methylimidazole (2-MeIm), 2,4-dimethylimidazole (2,4-diMeIm) and 2-aminobenzimidazole (2-AbzIm) have been used as the activating group.

Synthesis of activated monomers.

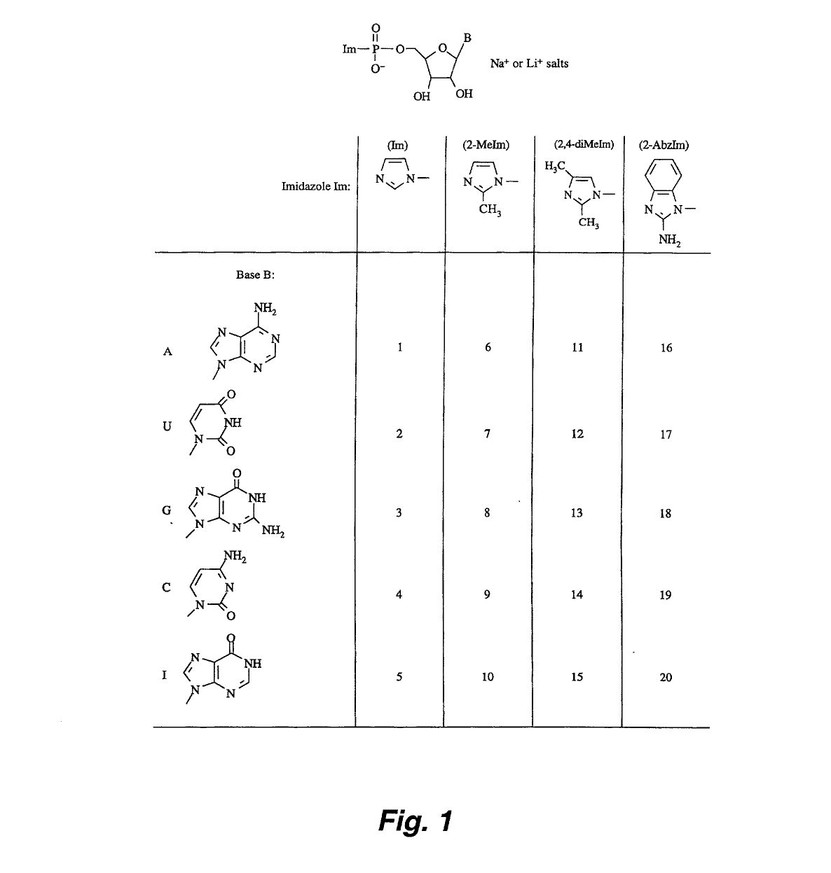

The corresponding phosphoimidazolide activated monomers (ImpN, 2-MeImpN, 2,4-diMeImpN and 2-AbzImpN where N=U, C, G, A or I; see structures in FIG. 1) were synthesized in our laboratory by oxidative condensation of the imidazolide and the nucleoside 5′monophosphate in anhydrous solvents. The monomer preparations were 96 to 98% pure and were obtained in practically quantitative yield. These nucleotide derivatives are solids, very stable under dry/cold conditions and relatively stable under dry/room temp. conditions.

The procedure for the synthesis of the sodium salt of 2-MeImpG is detailed below: 5′-GMP free acid, 0.46 g (1.25 mmol), and 1.64 g (20 mmol) of 2-methylimidazole were dissolved with stirring in 10 mL of dimethylsulfoxide (DMSO) in a 50 mL round bottom flask. Four hundred mL of freshly distilled triethylamine was added, followed by 1.05 g (4 mmol) of triphenylphosphine and 0.88 g (4 mmol) of 2,2′-dipyridyldisulfide. The mixture was stirred for 1 h under argon and quenched by adding it dropwise with stirring to a 250 mL Erlenmeyer flask cooled at 0° C. containing 50 mL of acetone, 50 mL of anhydrous ether, and 4 mL of a saturated sodium perchlorate/acetone solution. Sodium was replaced by lithium perchlorate for the synthesis of the corresponding lithium salt. The mixture was stirred for 20 min to allow precipitation and the product was collected by vacuum filtration and washed three times with 1:1 acetone: ether and three times with acetone. The product was dried under reduced pressure and purified if necessary by dissolving it in DMSO and reprecipitating as described above.

Template-directed RNA synthesis.

We initially used homopolymers as templates for the template-directed synthesis. These homopolymers were a mixture of chains 200 to 300 bases long. We have run reactions with the potassium salts of polycytidylic acid (poly(C)), polyadenylic acid (poly(A), polyguanylic acid (poly(G), polyuridylic acid (poly(U)) and polyinosinic acid (poly(I)) as well as with random sequence copolymers of poly(A,C,U), poly(A,G), poly(A,C) and poly(A,U). In all these cases in the presence of the complementary monomers and Mg²⁺/Pb²⁺ we obtained substantial amounts of oligomeric products. These homopolymer templates were used in place of the more preferable RNA oligomers, because they were commercially available at considerably less cost than RNA oligomers of specific sequence. In addition, the complementary products of the reactions using homopolymers are themselves homopolymers and thus relatively easy to identify. It is believed that the longer the template the more stable the double helical polymer/monomer complex and the more efficient the polymerization. This is why we believe that our methodology will be best suited for the synthesis of oligomers in the range of 30 to 100 bases long.

Each experiment with template, monomer and metal ion (T/M/Me) was run in concert with two controls, i.e. the template w/o the monomer (T/Me) and the monomer w/o the template (M/Me). The former was done in order to establish the extent of template degradation caused by the metal ions and the latter to investigate the extent of polymerization induced by the metal ions in the absence of a templating effect . Under the most preferred metal ion concentrations, the ribopolymers exhibited less than 10% degradation after 10 days at −18° C. In certain embodiments the volume of the initial solution ranged from 100 to 1000 microliters.

Analysis of Products

Samples were incubated under the conditions of the specific method for up to four weeks. Aliquots were obtained at regular intervals and analyzed as described below. RNase-free conditions were not used at this stage. Analysis of the reaction mixtures was done by high performance liquid chromatography (HPLC) using an RPC5 analytical column. This column was packed using a slurry packer in our lab with RPC5 material kindly provided by Leslie Orgel of the Salk Institute. RPC5 chromatography requires a gradient of NaClO4 conducted at pH 8 (with 2 mM tris.HClO4 as buffer) for most oligomers or at pH 12 (with 0.01 M NaOH) for the analysis of oligoguanylates. RPC5 is known to resolve oligomers as a function of length, base composition and internucleotide linkage isomerism. To the best of our knowledge, there is no other chromatography with more resolving power than RPC5 or even comparable to RPC5. However, a DNA-PAC 100 column by Dionex has specifications similar to RPC5.

A typical HPLC elution profile indicated that the monomer elutes early, the polymer elutes last and the oligomer products elute in between the monomer and the polymer peaks. The formation of a series of oligomer products with increasing length is a typical result of reactions run with the long homopolymers as templates (˜300 bases long). This is because initiation of the polymerization can occur at any site on the template and consequently the products vary in length. In order to produce the full complement one may accompany the synthesis step by an additional activation/ligation step as described earlier. Another system better suited for the synthesis of the complementary is a preformed hairpin with two arms, one short and one long that includes the template of interest. Synthesis in this system starts as elongation of the short arm that is faster than initiation at any other site in between. TD synthesis using hairpins has been applied successfully in solution to produce the full complement of sequences that include the other nucleotides besides uridine. Thus hairpin oligos may used to increase yield of the full complement. The hairpin can be subsequently removed from the product using other methods as described above.

Because products resulting from degradation of the template also elute in the same region as the polymerization products, careful controls and product identification are used to ensure that the peaks are the desired polymerization products rather than degradation products. With Mg²⁺/Pb²⁺ degradation of the templates is detectable at long incubation times.

This degradation can be suppressed by using conditions/parameters that expedite the polymerization reaction. Experiments suggest that the rate of polymerization is controlled by the pH of the aqueous solution before freezing or drying and by the imidazole derivative. Specifically, the reactivity follows the order Im→2-MeIn→2,4-diMeIm→2-AbzIm-. The optimal pH appears to be in the range 6.0<pH<7.0. In addition, the concentration of the metal ions is preferably kept at a minimum level, e.g. 0.004 M<[Mg²⁺]<0.008 M and 0.0005 M<[Pb²⁺]<0.002 M.

The following analytical methods were used or are recommended for the identification of the peaks eluting in the middle of the HPLC profile (polymerization or degradation products). These are (1) UV-vis spectroscopy using the diode-array detector of the HPLC instrument used for analysis. This was done with C/G samples, but not with U/A samples because of the similarity of the spectra of the latter pair. (2) NaOH degradation leads to cleavage of both internucleotide bonds yielding the corresponding monomer. The monomer was identified by analysis with C18 chromatography/diode array detection and coelution with standards using HPLC methods developed in our laboratory. NaOH degradation was done with isolated fractions of oligomer products. (3) Selective enzymatic degradation of the products with RNase A successfully distinguishes oligouridylates from poly(A) decomposition products and with Ribonuclease T1 oligoguanylates from poly(C) degradation products. Because the 2′-5′-internucleotide linkages can not be cleaved enzymatically, enzymatic degradation of the products with Nuclease P1 provided information about the extent of regioselectivity. (4) Mass spectroscopy can, but has not yet, been used to establish oligomer length from selected oligomer fractions.

It is believed that a monomer can be incorporated if the coding base on the template can form a hydrogen-bonded pair with it, may it be Watson-Crick or otherwise. In experiments with poly(U) as the template and mono(U) as the activated monomer, we observed some product formation, but much less efficient compared to the complementary pair. This result indicates that Watson-Crick base-pairing is favored under our conditions. Nevertheless in the absence of competition by the Watson-Crick complement, another base can be incorporated by our method if it forms a stable hydrogen-bonded pair with the base on the template, such as the U==U pair.

### Example 3

### Eutectic Phases in Ice Facilitate the Oligomerization of Phosphoimidazolide Activated Mononucleotides

In this example, we exploit ice-eutectic phases as a reaction medium for RNA synthesis. When an aqueous solution freezes, the solutes become concentrated in the spaces between the ice crystals. In principle, the increased concentration offsets the effect of the lower temperature and accelerates the reaction (Sanchez et al. (19660 Science 153: 72-73; Stribling and Miller (1991) J. Mol. Evol. 32: 289-295; Gryaznov and Letsinger (1993) J. Am. Chem. Soc. 115: 3808-3809; Riu and Orgel (1997) J. Am. Chem. Soc. 119: 4791-4792). In this example, we show that in the presence of metal ions in dilute solutions, frozen samples of phosphoimidazolide-activated uridine react within days at −18 ° C. to form oligouridylates up to 10 bases in length. The product yields typically exceed 90% and approximately 30% of the oligomers include one or more 3′-5′-linkages. Oligouridylate synthesis is notoriously difficult (Hill et al. (1993) Orig. Life Evol. Biosph. 23: 285-290), but these conditions also facilitate the oligomerization of the other three nucleobases. It is believed this is the first demonstration that freezing facilitates oligonucleotide synthesis.

It has not been an easy task to demonstrate plausible synthetic pathways to the polymers required for the origin of life. One problem is that the concentration of organic monomers in the early oceans was so dilute that synthetic reactions could not readily take place.⁶ A second difficulty is that organic compounds in solution are subject to decomposition, so that the rates of monomer and polymer synthesis must be sufficiently rapid to balance the rates of hydrolysis and other degradation pathways. Bada et al. (1994) Proc. Natl. Acad. Sci. USA 91 1248-1250, noted that hydrolytic degradation rates in aqueous solutions are correlated with temperature, and suggested a possible solution for this problem. Because lower luminosity of the sun four billion years ago may have resulted in partial freezing of the oceans, the resulting global low temperatures would clearly preserve organic compounds present in the environment. Assuming that reaction rates would be negligible in ice, it was proposed that synthetic reactions would take place in transient melts produced by large cometary and meteoritic impacts.

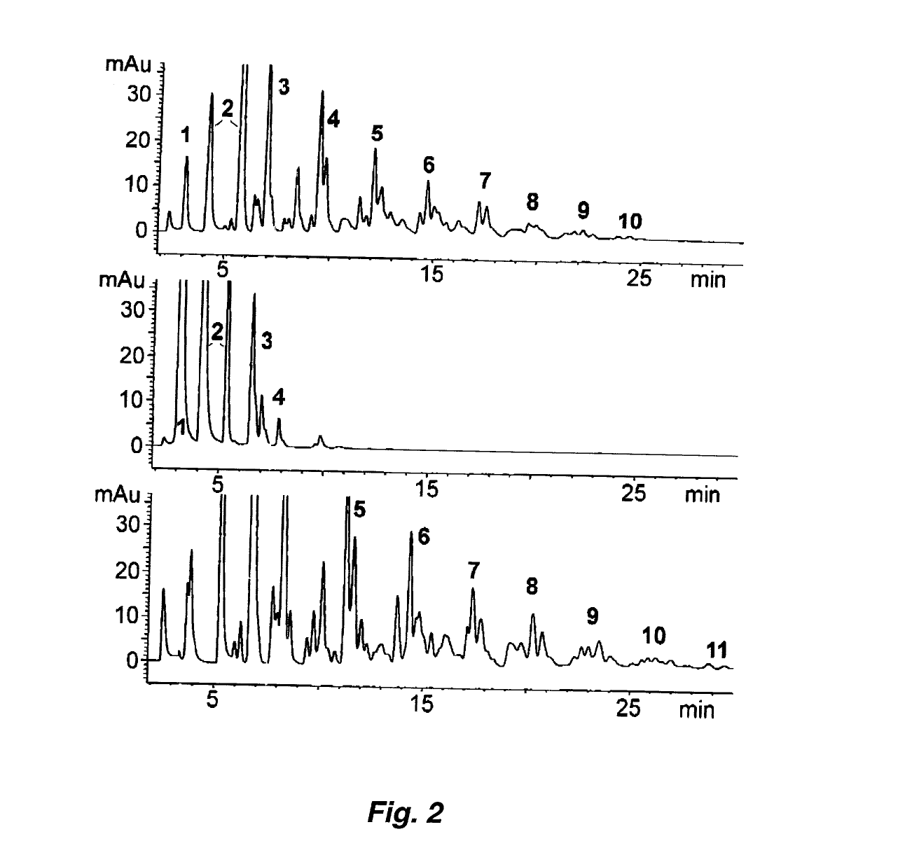

In the study reported here, we investigated the possibility that significant prebiotic reactions could occur in ice at subzero temperatures and in the absence of large scale melting. Because of the current interest in a probable RNA world (Gilbert (1986) Nature 319: 618), we chose to use monomers of RNA to explore ice as a reaction medium. In our initial experiments we made up solutions of ImpU, a phosphoimidazolide-activated uridine derivative (see legend, FIG. 2 for details). Incubation of the frozen (−18.3±0.1° C.) mixtures for 9 days led to partial consumption of the monomer and yielded oligouridylates up to 10 bases long as shown by high performance liquid chromatography (HPLC) (FIG. 2, top and 1^{st} entry in Table 2). We included magnesium/lead ions in our solutions because this specific metal ion pair was known to enhance polymerization in solution at 0° C. (Sawai (1976) J. Am. Chem Soc. 98, 7037-7039; Sleeper and Orgel (1979) J. Mol. Evol. 12, 357-364; Sleeper et al. (1979) J. Mol. Evol. 13: 203-214; Sawai and Ohno (1981) Chem. Pharm. Bull. 29: 2237-2245; Rohatgi et al. (1996) J. Am. Chem. Soc. 118: 3332-3339). These earlier reports indicated that concentrated solutions (50 mM in both nucleotide and lead ion) yield substantial amounts of oligomers up to 5 bases long (Sleeper and Orgel (1979) J. Mol. Evol. 12, 357-364; Sawai and Ohno (1981) Chem. Pharm. Bull. 29: 2237-2245).

Incubation of ImpU under otherwise similar conditions (see FIG. 2) but in the absence of lead yielded much less product composed of dimers, trimers and traces of tetramers (FIG. 2, middle). Incubation up to 31 days in the presence of Mg²⁺/Pb²⁺ resulted in practically quantitative conversion of the substrate to oligomers up to 11 bases long (FIG. 2, bottom and 2^{nd} entry in Table 2). Only 6.6% 5′UMP, the hydrolyzed substrate, was present, suggesting that ImpU hydrolysis was inhibited and that metal-ion facilitated degradation of oligomers was negligible under our conditions. Control experiments showed that in the liquid phase dilute ImpU solutions (5 mM) primarily yielded 5′-UMP as a hydrolysis product, and virtually no oligomers.

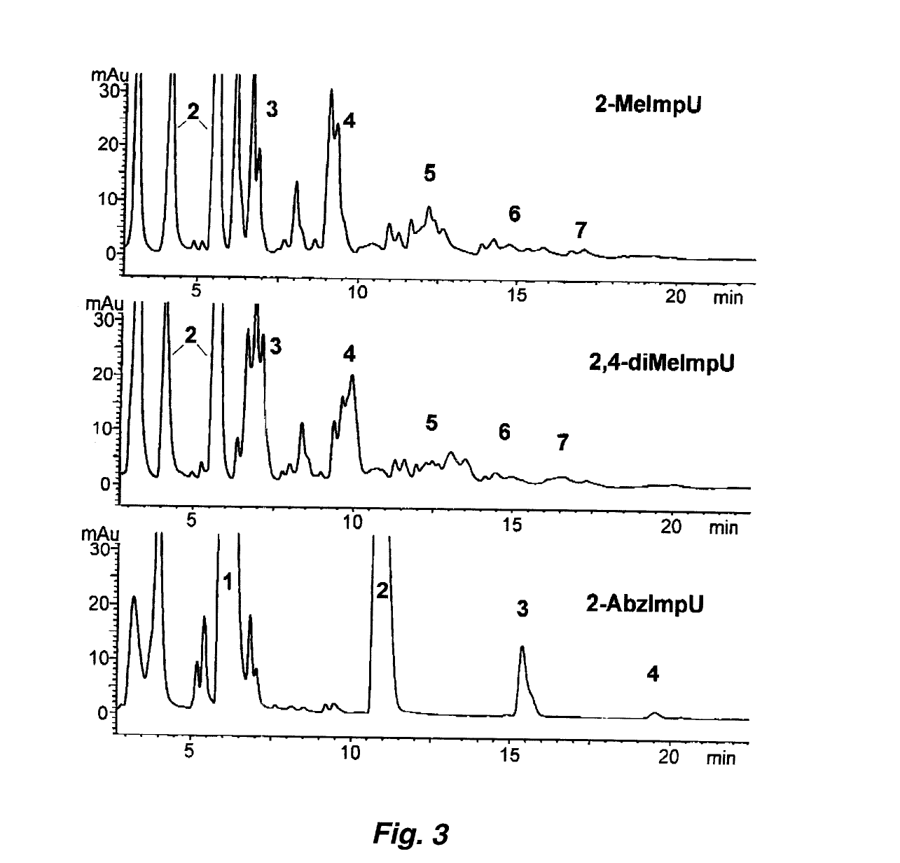

The effect of freezing is neither restricted to the specific activation or to the base itself. We explored the effect of the imidazolide activating group by comparing imidazole (ImpU), 2-methylimidazole (2-MeImpU), 2,4-dimethylimidazole (2,4-diMeImpU) and 2-aminobenzimidazole (2-AbzImpU). These derivatives show different reactivity under our conditions as they do in solution. FIG. 3 indicates that after 8 days at −18° C. the oligomeric product yield is similar for 2-MeImpU and 2,4-diMeImpU, but somewhat less than that obtained from ImpU (compare with FIG. 2, top, Table 2). ImpU forms longer oligomers faster than 2-MeImpU and 2,4-diMeImpU. Comparison of the reaction of ImpU as a function of time with that of 2-MeImpU indicates that oligomer yields are already high within 8-9 days of incubation and increase with prolonged incubation (Table 2). 2-AbzImpU reacts slowest and produces oligomers up to the tetramer (FIG. 3, bottom) which elute late because they still carry the 2-aminobenzimidazole moiety as shown by their UV-vis spectra. Even after 4 weeks half of the starting material, 2-AbzImpU, is intact under these conditions.

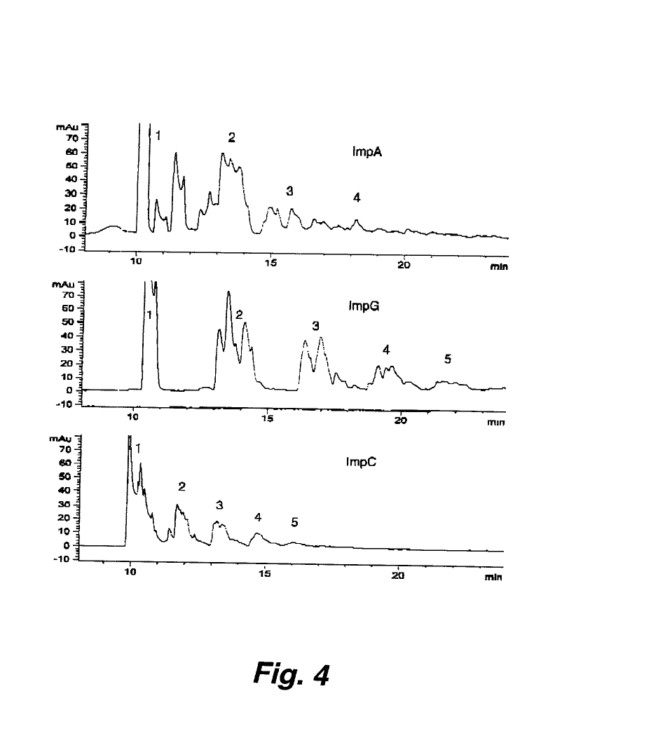

The effect of the nucleobase on polymerization was tested by carrying out experiments with phosphoimidazolide-activated derivatives of cytidine (ImpC), adenosine (ImpA) or guanosine (ImpG) in the presence of Mg²⁺/Pb²⁺; conditions are given in the caption of FIG. 4. We found that these substrates react within a week to yield approximately 80% of oligomers 4 to 6 bases long (FIG. 4). The absence of lead has the same effect with these substrates as it did with ImpU. The efficiency obtained with C, G and A bases is less than the efficiency seen with U (note: three features of a polymerization are of interest here: (I) the yield, i.e. the percent of monomer that becomes incorporated in oligomers; (ii) the extent of oligomerization, i.e. the longest detectable oligomer and (iii) the regioselectivity expressed as the percent of internucleotide linkages that are 3′-5′). This is surprising since U is the least efficient base in oligomerization reactions carried out in solution (Hill et al. (1993) Orig. Life Evol. Biosph. 23: 285-290; Wu and Orgel (1992) J. Am. Chem. Soc. 114, 7963-7969).

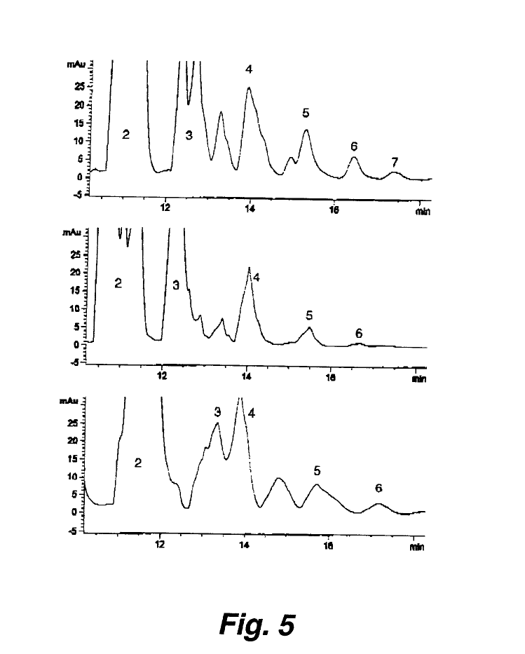

To show that the combination of Mg²⁺/Pb²⁺ is not unique (Rohatgi et al. (1996) J. Am. Chem. Soc. 118: 3332-3339) we tested other metal ions or pairs of metal ions, such as Pb²⁺ (as Pb(NO₃)₂, not shown) alone, Mn²⁺ (as MnCl₂, not shown), Mn²⁺/Pb²⁺ (FIG. 5, middle) Sn²⁺ (as SnCl₂, not shown) and Mg²⁺/Sn²⁺ (FIG. 5, bottom) which enhanced polymerization in the order Pb²⁺≈Mg²⁺/Pb²⁺≈Mn²⁺/Pb²⁺>Sn²⁺≈Mg²⁺/Sn²⁺>>Mg²⁺≈Mn²⁺. We found that Mn²⁺, like Mg²⁺, exhibits a small catalytic effect whereas Mn²⁺/Pb²⁺, just like Mg²⁺/Pb²⁺, exhibits a substantial effect. Tin was tested because it is in the same group of the periodic table as lead. Both Sn²⁺ and Mg²⁺/Sn²⁺ are excellent oligomerization catalysts. In view of the fact that Pb²⁺ does not act as catalyst in the absence of Mg²⁺ in solution reactions (Sleeper and Orgel (1979) J. Mol. Evol. 12, 357-364; Sleeper et al. (1979) J. Mol. Evol. 13: 203-214; Rohatgi et al. (1996) J. Am. Chem. Soc. 118: 3332-3339), it was surprising to find that Pb²⁺ alone exhibits in ice a catalytic effect practically as strong as Mg²⁺/Pb²⁺. Interestingly, catalysis of oligomerization occurs in the presence of the chloride salts, such as MgCl₂ (FIG. 5, top) and SnCl₂ that typically precipitate lead ion. Increasing the solution concentration of either metal ion resulted in faster oligomerization but also faster degradation of the products as demonstrated by control experiments with homopolymers such as poly(U), poly(C) and poly(A).

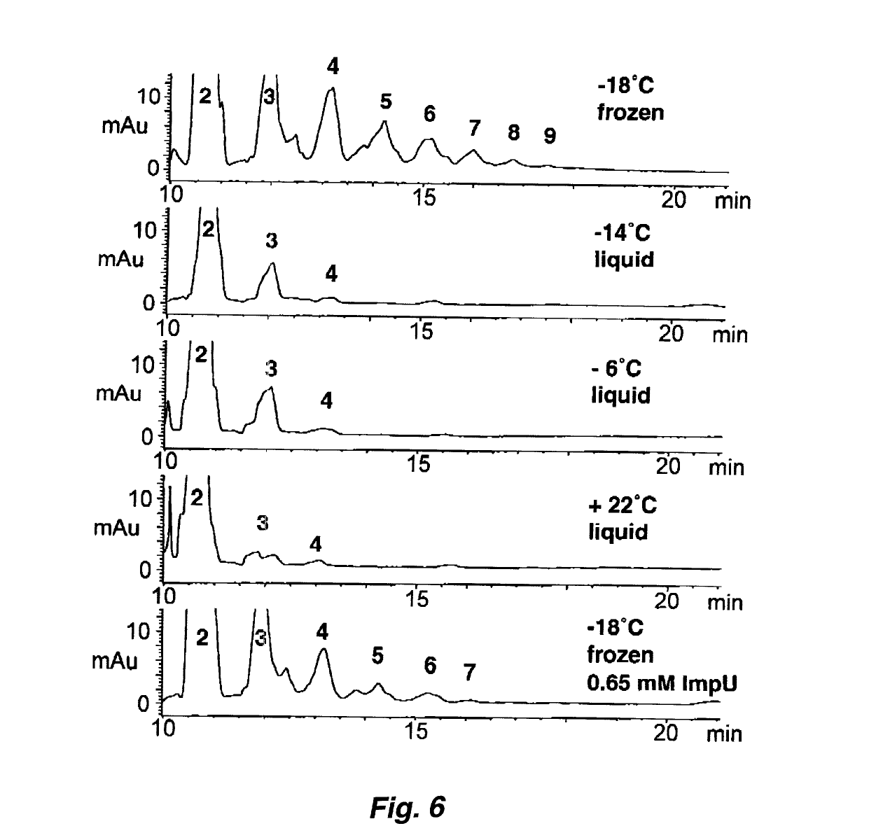

To confirm that the frozen state is a necessary condition for enhanced oligomerization and high efficiency we carried out control experiments in liquid solutions with ImpU as described in the caption of FIG. 6. We eliminated the effect of temperature on pH by conducting reactions in the range 6<pH<8 by addition of 11 mM MES or HEPES buffer at the appropriate pH and by showing that polymerization was comparably favored in this pH range. A series of identical samples was prepared and placed at −18, −14, −6 and +22° C. Typically the samples at −18° C. were frozen and the others were liquid. FIG. 6 (top profile) indicates that after 4.8 days of incubation at −18° C. oligouridylates up to the 9-mer are formed with 80% total yield based on initial monomer. In contrast, at −14° C. (liquid) the corresponding yield is only 20% and oligomer products are composed of dimers, trimers and traces of tetramers (FIG. 6, 2^{nd} from the top). Some −14° C. samples accidentally froze; they showed similar results as the frozen −18° C. samples. Oligomerization is also suppressed at the higher temperatures. For example, oligomer yields are reduced to about 20% at −6° C. (FIG. 6, 3^{rd} from the top) and at 22° C. (FIG. 6, 4^{th} from the top). Longer incubation did not afford more product in the liquid phase.

Lowering the initial concentration of ImpU did not substantially reduce oligomerization efficiency in the frozen mixtures. This was shown by carrying out experiments with ImpU concentration in the range 0.65 mM≦[ImpU]≦5.2 mM. For example, a sample of 0.65 M ImpU (FIG. 6, bottom profile) incubated under the same conditions as a sample of 5.2 mM ImpU (FIG. 6, top profile) exhibits comparable efficiency, i.e. oligomerization up to the 8-mer after 4.8 days of incubation, despite an eight-fold lower concentration. It is important to note that typical concentrations of materials in non-enzymatic polymerizations include 50 mM activated nucleotide, 25 mM lead nitrate and 150 mM magnesium nitrate, whereas the concentrations used in this study are 5±1.5 mM, 1.0±0.5 mM and 5.0±1.2 mM, respectively. As mentioned above, efficient oligomerizations in ice were obtained with a concentration of ImpU as low as 0.65 mM. It follows that chemistry in the frozen state has a 10- to 80-fold advantage in nucleotide starting material concentration and a 20 to 30-fold advantage in metal ion concentration over the chemistry in solution.

To assess the regioselectivity in terms of the percent of 3′-5′ linkages in the products, selected samples were digested with ribonuclease A and alkaline phosphatase. The samples before and after digestion were analyzed with HPLC/RPC5 chromatography in order to identify the peaks that disappear after digestion. Ribonuclease A cleaves selectively 3′-5′ linkages, while alkaline phosphatase cleaves free 5′ phosphate groups, but leaves intact any pyrophosphate-capped and cyclic oligomers. We carried out the enzymatic digestions in reaction mixtures made of 5.2 (or 2.6) mM ImpU, 5.2 mM Mg²+and 1.3 mM Pb²+at pH 6.55. These mixtures were incubated for a period of 27 to 32 days at −18° C. before enzymatic digestion to ensure that most of the imidazolide derivatives had hydrolyzed. Based on the enzymatic degradation pattern we concluded that the main products are linear internucleotide-linked isomers; minor products include pyrophosphate capped and/or cyclic oligomers. Ribonuclease A digestion allowed an estimate of 30±5% of products which carry one or more 3′-5′-linkages.

The 30±5% of oligomers carrying one or more 3′-5′ linkages obtained under our conditions indicates a higher degree or regioselectivity for the natural linkage than the less than 10% of such linkages reported for oligomerization in solution (Sleeper and Orgel (1979) J. Mol. Evol. 12, 357-364). This result suggests that, compared to dilute nucleotide solutions, the concentrated solutions in the frozen state modify the ratio of 2′-5′- to 3′-5′-linked isomers in favor of the natural isomers. There is precedent for such a concentration effect in oligomerizations carried out at room temperature with mononucleotide concentration in the range of 0.1 to 1.0 M in the presence of high concentration of Mg²⁺ or Mn²⁺ (Kanavarioti (1997) Origins Life Evol Biosph, 27: 357-376; Kanavarioti et al. (1999) Origins Life Evol. Biosph. 29: 473-487). An additional explanation for the higher fraction of RNA linkages observed at −18° C. compared to 0° C.¹⁴ is that the frozen state imposes a stricter orientation within the reactive complex that brings the 3′-OH closer to the P—N bond.

Mechanistic questions about the polymerization cannot be answered directly with these preliminary results. Nevertheless, the fact that the reaction in frozen samples is metal-ion catalyzed and that the reactivity rank of the imidazolide derivatives parallels their reactivity in solution (Rohatgi et al. (1996) J. Am. Chem. Soc. 118: 3332-3339; Kanavarioti, et al. (1989) J. Am. Chem. Soc., 111, 7247-7257) strongly suggest a similar mechanism for the liquid and for the frozen phase. Polymerization with frozen mixtures most likely takes place in cavities between ice crystals filled with liquid eutectics of concentrated solutes (Gryaznov and Letsinger (1993) J. Am. Chem. Soc. 115: 3808-3809), and not by adsorption of the chemicals on ice. As to the hydrolysis of the oligomers, the very small degree of oligomer degradation indicates that this reaction is inhibited in the frozen state. This must be the result of two factors. One is that hydrolysis being a first-order reaction it does not benefit from the concentrating effect of the frozen state; the other is the low temperature that reduces its rate.

Efficient oligouridylate synthesis and/or uridine incorporation in polynucleotides is a requirement for the advent of an RNA world (Gilbert (1986) Nature 319: 618;Joyce and Orgel (1993) pages 1-25 In: The RNA World (eds Gesteland, R. F. & Atkins, J.

F.), Cold Spring Harbor Lab. Press, Cold Spring Harbor, N.Y.). An RNA or RNA-like population would likely have included two or more pairs of complementary nucleobases because one pair limits information content. Uracil has been detected in samples of the Murchison meteorite (Stocks and Schwartz (1979) Nature 282: 709-710) and can be synthesized in good yield under simulated prebiotic conditions (Robertson and Miller (1995) Nature 375, 772-774). Although a satisfactory synthesis of the corresponding nucleoside, uridine, is elusive (Joyce and Orgel (1993) pages 1-25 In: The RNA World (eds Gesteland, R. F. & Atkins, J. F.), Cold Spring Harbor Lab. Press, Cold Spring Harbor, N.Y.) syntheses for 5′-uridylic acid (Reimann and Zubay (1999) Origins Life Evol Biosph. 29: 229-247) and 5′-phosphoimidazolide activated uridylic acid (Lohrmann (1977) J. Mol. Evol. 10, 137-154) from uridine derivatives are available. The importance of uridine has been underlined with experiments suggesting that 5-substituted uracils could act as a bridge between the RNA world and the DNA-protein world (Robertson and Miller (1995) Science 268: 702-705). So far there are no adequate prebiotically plausible syntheses for the formation of medium size 3′-5′-linked oligouridylates in the range 5 to 10-bases long. Such medium size oligouridylates can not be synthesized by other means because uridine resists incorporation in non-enzymatic template-directed polynucleotide syntheses (Hill et al. (1993) Orig. Life Evol. Biosph. 23: 285-290; Wu and Orgel(1992) J. Am. Chem. Soc. 114: 7963-7969) and oligouridylates shorter than pentamers are inefficiently ligated even in the presence of the complementary polynucleotide (Sawai and Wada (2000) Origins Life Evol Biosph. 30: 503-511). In contrast, oligouridylates as long or longer than pentamers can be efficiently ligated in the presence of an oligoadenylate. Efficient non-enzymatic synthesis of medium size—5 to 10 bases long—oligouridylates would strongly support the RNA world hypothesis.

Freezing and eutectic phase concentration offers a partial solution to the lack of prebiotic methods for the synthesis of oligouridylates 5 to 10 bases long. Three conditions are known to facilitate oligouridylate synthesis in prebiotic simulations. The first is with Mg²⁺/Pb²⁺ in solution, a reaction with modest yield that produces oligouridylates up to the 5-mer that are predominantly 2′-5′-linked (Sleeper and Orgel (1979) J. Mol. Evol. 12, 357-364; Sawai and Ohno (1981) Chem. Pharm. Bull. 29: 2237-2245). The second condition involves uranyl oxide nitrate that catalyzes the synthesis of oligouridylates up to 12-bases long, albeit predominantly 2′-5′-internucleotide linked isomers (Sawai et al. (1989) Bull. Chem. Soc. Jpn. 62: 2018-2023). These two conditions do not yield medium size 3′-5′-linked oligouridylates. Last but not least montmorillonite clay exhibits a substantial catalytic effect on nucleotide polymerization. It has been postulated that this particular clay served as the stepping stone between prebiotic chemistry and an RNA-world, because it produces substantial amounts of 3′-5′-linked oligomers (Ding et al. (1996) Origins Life Evol. Biosph. 26, 151-171; Kawamura and Ferris (1999) Origins Life Evol. Biosph. 29, 563-591). The 30±5% of oligomers containing a preponderance of 3′-5′-linkages determined under our conditions is slightly better or comparable to the regioselectivity (24%) reported for ImpU oligomerization in the presence of montmorillonite clay (Ding et al. (1996) Origins Life Evol. Biosph. 26, 151-171; Kawamura and Ferris (1999) Origins Life Evol. Biosph. 29, 563-591). However, montmorillonite-induced ImpU oligomerization yields, at most, 6.1% of oligomers as long or longer than the 5-mer (Table 2, last two entries). Only in the frozen state are substantial amounts—up to 35.6% (2^{nd} entry)—of oligouridylates as long or longer than the pentamer formed. These materials could successfully become incorporated in template-directed ligations (Sawai and Wada (2000) Origins Life Evol Biosph. 30: 503-511). It is possible that oligomerization in the presence of uranyl oxide, montmorillonite or a preformed polynucleotide acting as template could be further improved by eutectic phase concentration.

The lack of high yield prebiotic reactions (Joyce and Orgel (1993) pages 1-25 In: The RNA World (eds Gesteland, R. F. & Atkins, J. F.), Cold Spring Harbor Lab. Press, Cold Spring Harbor, N.Y.; Lazcano and Miller (1996) Cell 85, 793-798) leading to synthesis of biochemically relevant building blocks suggests that solutions of organic compounds on the early Earth were likely to have been highly dilute. In order for polymer synthesis to occur, concentrating mechanisms would be required, and eutectic phases present in frozen mixtures provide a testable hypothesis. The lower luminosity of the young sun must have provided both Early Earth and Mars with a thick layer of ice covering the oceans. Assuming that the building blocks were available, the ice layer could have provided a broad niche for prebiotic syntheses at subzero temperatures both on early Earth and on Mars. The fact that RNA synthesis from activated nucleotides readily takes place in a frozen mixture suggests that impact events were not necessarily required for significant prebiotic polymerization reactions to occur.

TABLE 2
Percent yield of oligouridylate products under various conditions. The length of oligouridylate products
is identified by numbers in italics. Isomers of the same length are counted together. Substrate 1 stands
for ImpU and 2 for 2-MeImpU. Conditions for the first two entries are given in the caption of FIG 2.
Conditions for the third and fourth entries are given in the caption of FIG. 3. Also note that all the
reactions in this work were carried out in 1.7 ML microcentrifuge tubes and reaction mixtures were
200 ± 30 μL. The last two entries refer to reactions carried out with 15 mM ImpU in the presence
of 75 mM Mg²⁺ and 50 mg montmorillonite clay. Yield of an oligomer in percent is the HPLC area under
the indicated oligomer peak over the total HPLC area that corresponds in the initial monomer. Under 1-
are included both the activated as well as the hydrolyzed monomer. 2-include all dimers, i.e. 2′-5′-
linked, 3′-5′-linked, pyrophosphate-linked and their imidazolide derivatives. Length of oligomer in this
work was estimated by comparison of retention times of peaks from partially degraded poly(U) and
the peaks for our samples run with the same chromatography.

| | temp/inc | | | | | | | | | | | | |
|---|---|---|---|---|---|---|---|---|---|---|---|---|---|
| subst | ° C./days | 1- | 2- | 3- | 4- | 5- | 6- | 7- | 8- | 9- | 10- | 11- | reference |
| 1 | −18/9 | 12 | 28 | 16.5 | 16.6 | 10.4 | 4.8 | 4.6 | 2.5 | 1.1 | 0.4 | — | this example |
| 1 | −18/31 | 6.6 | 25.8 | 18.4 | 13.1 | 10.4 | 9.5 | 7.0 | 4.5 | 2.8 | 1.1 | 0.3 | this example |
| 2 | −18/8 | 24.4 | 38.4 | 16.8 | 12.0 | 4.4 | 1.1 | 1.0 | — | — | — | — | this example |
| 2 | −18/28 | 5.5 | 34.0 | 13.3 | 16.7 | 14.3 | 6.1 | 5.4 | 3.1 | 1.4 | 0.5 | — | this example |
| 1 | 23/7 | 72 | 21 | 2.6 | 0.6 | 0.2 | 0.1 | — | — | — | — | — | Ding et al. (1996) Origins Life |
| | | | | | | | | | | | | | Evol. Biosph. 26: 151-171 |
| 1 | 25/7 | 30 | 42 | 12 | 6.2 | 3.3 | 1.8 | 1.0 | — | — | — | — | Kawamura and Ferris(1999) |
| | | | | | | | | | | | | | Origins Life Evol. Biosph. 29: |
| | | | | | | | | | | | | | 563-591 |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of synthesizing a ribonucleic acid, said method comprising contacting a template nucleic acid, in the presence of lead ions (Pb²⁺) or tin (Sn²⁺) ions, with a nucleotide derivatized with an imidazolide.

2. The method of claim 1, wherein said contacting is in the presence of tin ions.

3. The method of claim 1, wherein said contacting is in the presence of lead ions.

4. The method of claim 1, wherein said contacting is in an aqueous solution.

5. The method of claim 4, wherein said contacting comprises withdrawing water from a complex formed between said nucleotide and said template nucleic acid.

6. The method of claim 5, wherein said withdrawing comprises drying said aqueous solution under a vacuum.

7. The method of claim 5, wherein said withdrawing comprises freezing said aqueous solution.

8. The method of claim 1, wherein said nucleotide is a ribonucleoside 5′-monophosphate.

9. The method of claim 8, wherein said nucleotide is selected from the group consisting of cytidylic acid, adenylic acid, guanylic acid, uridylic acid, and inosinic acid.

10. The method of claim 1, wherein said nucleotide is a deoxyribonucleoside 5′-monophosphate.

11. The method of claim 1, wherein said imidazolide is selected from the group consisting of imidazole (Im), 2-methylimidazole (2-MeIm), 2,4-dimethylimidazole (2,4-diMeIm), 2-aminobenzimidazole (2-aminobzIm), and 2,4,5-trimethylimidazole (2,4,5-triMeIm).

12. The method of claim 1, wherein said tin ions or lead ions are present at a concentration ranging from about 0.0005 M to about 0.004M.

13. The method of claim 12, wherein said tin or lead is present at a concentration of about 0.0015 M.

14. The method of claim 1, wherein the derivatized nucleic acid is also contacted with magnesium (Mg²+) or manganese (Mn²+) ions.

15. The method of claim 14, wherein said magnesium or manganese is present at a concentration ranging from about 0.0001 to about 0.010 M.

16. The method of claim 15, wherein said magnesium or manganese is present at a concentration of about 0.005 M.

17. The method of claim 14, wherein said magnesium or manganese is provided as a magnesium or manganese salt.

18. The method of claim 17, wherein said magnesium or manganese salt is not a chloride salt.

19. The method of claim 18, wherein said magnesium or manganese salt is magnesium nitrate or manganese nitrate.

20. The method of claim 1, wherein said lead is provided as a lead salt.

21. The method of claim 20, wherein said lead salt is not a chloride salt.

22. The method of claim 21, wherein said lead salt is lead nitrate.

23. The method of claim 1, wherein a ratio of template nucleic acid to mononucleotide to metal ion is about 1:1:1 where the amount of template is expressed in monomer equivalents.

24. The method of claim 14, wherein a ratio of Mg²⁺ to Pb²⁺ is about 4:1.

25. The method of claim 1, wherein said template ranges in length from about 30 to about 300 nucleotides.

26. The method of claim 1, wherein
said nucleotide comprises a cytodine, an adenine, a guanosine, an inosine, and a uradine;
said contacting is in an aqueous solution;
said tin or lead is present at a concentration of about 0.0015 M;
said magnesium is present at a concentration of about 0.005 M; and
said template nucleic acid is present at a concentration of about 0.005M expressed in monomer equivalents.

27. The method of claim 26, wherein said tin or lead is tin.

28. The method of claim 26, wherein said tin or lead is lead.

29. The method of claim 26, further comprising freezing the mixture.

30. The method of claim 29, wherein said mixture is frozen for about 5 to about 30 days.

31. The method of claim 26, further comprising drying the mixture.

32. The method of claim 31, wherein the dried mixture is stored for about 2 to about 6 days.

33. A method of synthesizing a ribonucleic acid, said method comprising contacting, in the presence of lead ions (Pb²⁺) or tin (Sn²⁺) ions, a plurality of activated nucleotides wherein said activated nucleotides are nucleotides derivatized with an imidazolide.

34. The method of claim 33, wherein said contacting is in the presence of tin ions.

35. The method of claim 33, wherein said contacting is in the presence of lead ions.

36. The method of claim 33, wherein said contacting is in an aqueous solution.

37. The method of claim 36, wherein said contacting comprises withdrawing water from a complex formed between said nucleotides.

38. The method of claim 37, wherein said withdrawing comprises drying said aqueous solution under a vacuum.

39. The method of claim 37, wherein said withdrawing comprises freezing said aqueous solution.

40. The method of claim 33, wherein said nucleotides are ribonucleoside 5′-monophosphates.

41. The method of claim 40, wherein said nucleotides are independently selected from the group consisting of cytidylic acid, adenylic acid, guanylic acid, uridylic acid, and inosinic acid.

42. The method of claim 33, wherein said nucleotides are deoxyribonucleoside 5′-monophosphates.

43. The method of claim 33, wherein said imidazolide is selected from the group consisting of imidazole (Im), 2-methylimidazole (2-MeIm), 2,4-dimethylimidazole (2,4-diMeIm), 2-aminobenzimidazole (2-aminobzIm), and 2,4,5-trimethylimidazole (2,4,5-triMeIm).

44. The method of claim 33, wherein said tin or lead is present at a concentration ranging from about 0.0005 M to about 0.004M.

45. The method of claim 12, wherein said tin or lead is present at a concentration of about 0.0015 M.

46. The method of claim 33, wherein the derivatized nucleic acid is also contacted with magnesium (Mg²⁺) or manganese (Mn²⁺) ions.

47. The method of claim 46, wherein said magnesium or manganese is present at a concentration ranging from about 0.0001 to about 0.010 M.

48. The method of claim 47, wherein said magnesium or manganese is present at a concentration of about 0.005 M.

49. The method of claim 46, wherein said magnesium or manganese is provided as a magnesium or manganese salt.

50. The method of claim 49, wherein said magnesium or manganese salt is not a chloride salt.

51. The method of claim 50, wherein said magnesium or manganese salt is magnesium nitrate or manganese nitrate.

52. The method of claim 33, wherein said lead is provided as a lead salt.

53. The method of claim 52, wherein said lead salt is not a chloride salt.

54. The method of claim 53, wherein said lead salt is lead nitrate.

55. The method of claim 33, wherein a ratio of mononucleotide to metal ion is about 1:1.

56. The method of claim 46, wherein a ratio of Mg²⁺ to Pb²⁺ is about 4:1.

57. The method of claim 33, wherein
said nucleotide comprises a cytodine, an adenine, a guanosine, an inosine, and a uradine;
said contacting is in an aqueous solution;
said tin or lead is present at a concentration of about 0.0015 M; and
said magnesium is present at a concentration of about 0.005 M.

58. The method of claim 57, wherein said tin or lead is tin.

59. The method of claim 57, wherein said tin or lead is lead.

60. The method of claim 57, further comprising freezing the mixture.

61. The method of claim 60, wherein said mixture is frozen for about 5 to about 30 days.

62. The method of claim 57, further comprising drying the mixture.

63. The method of claim 57, wherein the dried mixture is stored for about 2 to about 6 days.

64. A kit for the synthesis of a ribonucleic acid, said kit comprising:
a container containing a nucleotide derivatized with an imidazolide; and
a container containing a lead salt or a tin salt.

65. The kit of claim 64, wherein said kit further comprises a container containing a magnesium salt.

66. The kit of claim 65, wherein said container containing a magnesium salt and said container containing a lead salt are the same container.

67. The kit of claim 66, wherein said lead salt and said magnesium salt are in an aqueous solution.

68. The kit of claim 64, wherein said magnesium salt is not a chloride salt.

69. The kit of claim 64, wherein said lead salt is not a chloride salt.

70. The kit of claim 64, wherein said lead salt is lead nitrate.

71. The kit of claim 64, wherein said magnesium salt is magnesium nitrate.

72. The kit of claim 64, wherein said kit further comprises instructional materials describing the synthesis of RNA according to the method of claim 1.
